(19) United States
(12) Patent Application Publication
(10) Pub. No.: US 2026/0053880 A1
(43) Pub. Date: Feb. 26, 2026

(54) Nrf2 activator and method for producing same

(71) Applicants: NATIONAL INSTITUTE OF ADVANCED INDUSTRIAL SCIENCE AND TECHNOLOGY, Tokyo (JP); OKURA INDUSTRIAL CO., LTD., Kagawa (JP)

(72) Inventors: Ryo MIYATA, Ibaraki (JP); Yousuke TABEI, Ibaraki (JP); Hiroko ABE, Ibaraki (JP); Yoshihiro NAKAJIMA, Ibaraki (JP); Yoshiki TAKAISHI, Kagawa (JP); Syunryo AZUMA, Kagawa (JP); Shiori FUKUZAKI, Kagawa (JP); Kana UTSUMI, Kagawa (JP)

(21) Appl. No.: 19/304,604

(22) Filed: Aug. 20, 2025

(30) Foreign Application Priority Data
Aug. 21, 2024 (JP) 2024-139792

(51) Int. Cl. A61K 36/63

(52) U.S. Cl. CPC A61K 36/63; A61K 2236/331; A61K 2236/37; A61K 2236/51

(57) ABSTRACT

The present invention provides a novel Nrf2 activator that is derived from a natural component and that has a high Nrf2 activation ability. The invention also provides a novel treatment agent for a condition treatable by Nrf2 activation, the treatment agent being derived from a natural component having a high Nrf2 activation ability. Specifically, provided are a treatment agent for a condition treatable by Nrf2 activation, the treatment agent comprising a pressurized hot water extract of olive, and an Nrf2 activator comprising a pressurized hot water extract of olive.

### TECHNICAL FIELD

The present invention relates to an Nrf2 activator derived from a natural component and a method for producing the Nrf2 activator.

### Background Art

NF-E2-related factor 2 (Nrf2) is a transcriptional regulatory factor having a basic leucine zipper structure. Nrf2 is also a transcription factor that becomes activated in response to stress, such as oxidative stress. It is known that Nrf2 functions to increase the gene expression of antioxidant enzymes, detoxification metabolic enzymes, and the like, and reduce the gene expression of cytokines that induce inflammation. Since there have also been reports that activation of Nrf2 can ameliorate the pathology of Alzheimer's disease, there is a strong demand for the development of methods for activating this factor.

### Citation List

### Patent Literature

PTL 1: JP2022-174199A

### Non-patent Literature

NPL 1: Molecular and Cellular Biology, DOI: 10.1128/MCB.00467-19

### SUMMARY OF INVENTION

### Technical Problem

An object of the present invention is to provide a novel Nrf2 activator that is derived from a natural component and that has a high Nrf2 activation ability. In another embodiment, an object of the present invention is to provide a novel treatment agent for a condition treatable by Nrf2 activation, the treatment agent being derived from a natural component having a high Nrf2 activation ability.

### Solution to Problem

Under such circumstances, the present inventors have conducted extensive research and found that an extract obtained by subjecting olive to pressurized hot water extraction has an unexpectedly high Nrf2 activation ability. The present invention is based on this novel finding.

Accordingly, the present invention provides the following items:

### Item 1.

A treatment agent for a condition treatable by Nrf2 activation, comprising a pressurized hot water extract of olive.

### Item 2.

An Nrf2 activator comprising a pressurized hot water extract of olive.

### Item 3.

The agent or activator according to Item 1 or 2, wherein the pressurized hot water extract of olive comprises a pressurized hot water extract of olive leaves.

### Item 4.

The agent or activator according to any one of Items 1 to 3, wherein the extract is obtained by subjecting olive to pressurized hot water extraction at a temperature of 150° C. or higher.

### Item 5.

The agent or activator according to Item 4, wherein in the pressurized hot water extraction, the extraction pressure is greater than 0.1 MPa and 5.0 MPa or less, and the extraction time is 120 minutes or less.

### Item 6.

A method for producing a treatment agent for a condition treatable by Nrf2 activation, the method comprising a step of subjecting a mixture of olive and an extraction solvent to pressurized hot water extraction.

Item 7.

A method for producing an Nrf2 activator, comprising a step of subjecting a mixture of olive and an extraction solvent to pressurized hot water extraction.

Item 8.

The production method according to Item 6 or 7, wherein the olive is an olive leaf.

Item 9.

The production method according to any one of Items 6 to 8, wherein the pressurized hot water extraction step is performed at a temperature of 150° C. or higher.

Item 10.

The production method according to Item 9, wherein the pressurized hot water extraction step is performed at a pressure greater than 0.1 MPa and 5.0 MPa or less for 120 minutes or less.

### Advantageous Effects of Invention

The present invention can provide a novel Nrf2 activator that is derived from a natural component and that has a high Nrf2 activation ability while maintaining low cytotoxicity. In another embodiment, the present invention can provide a novel treatment agent for a condition treatable by Nrf2 activation, the treatment agent being derived from a natural component having a high Nrf2 activation ability while maintaining low cytotoxicity. As demonstrated in the Examples below, the present invention can achieve an extremely high Nrf2 activation ability while maintaining low cytotoxicity, compared with cases in which a hot water extract of olive or an ethanol extract of olive is used. Additionally, as demonstrated in the Examples described below, the present invention achieves a higher level of Nrf2 activation ability than can be expected from the Nrf2 activation ability of hydroxytyrosol and the Nrf2 activation ability of hydroxytyrosol acetate.

### BRIEF DESCRIPTION OF DRAWINGS

FIG. 1 shows the measurement results of Nrf2 activation and cell viability in Example 1. The graphs show Nrf2 activation (upper row) and cell viability (lower row) of a pressurized hot water extract of olive leaves (left), a 80% ethanol extract (center), and a hot water extract (right).

FIG. 2 shows the measurement results of Nrf2activation and cell viability in Example 2. The graphs show Nrf2 activation (upper row) and cell viability (lower row) of three types of pressurized hot water extracts of olive leaves obtained under different extraction conditions: (1) 160° C., 60 minutes (left); (2) 190° C., 30 minutes (center); and (3) 220° C., 15 minutes (right).

FIG. 3 shows quantitative values of the main components in the pressurized hot water extract of olive leaves, 80% ethanol extract, and hot water extract. The quantitative values for each compound are expressed in μg/mg of extract, and n.d. denotes below the detection limit.

FIG. 4 shows the results of Nrf2 activation (upper row) and cell viability (lower row) of the main components (1-5) contained in the pressurized hot water extract of olive leaves.

FIG. 5 shows a comparison among the contribution rates, to Nrf2 activation, of the compounds contained in the three types of pressurized hot water extracts of olive leaves obtained under different extraction conditions ((1) 160° C. for 60 minutes, (2) 190° C. for 30 minutes, and (3) 220° C. for 15 minutes).

### DESCRIPTION OF EMBODIMENTS

### Treatment Agent

In one embodiment, the present invention provides a treatment agent for a condition treatable by Nrf2 activation, the treatment agent comprising a pressurized hot water extract of olive.

In the present invention, the term “condition” in the phrase “a condition treatable by Nrf2 activation” may encompass not only so-called “diseases,” but also non-normal conditions that represent a preliminary stage of diseases.

In addition, in the present invention, the term “treatment” encompasses not only the therapy of a subject already in such a condition, but also the prevention of such a condition.

It is known that Nrf2 activation contributes to the elimination of reactive oxygen species and to the impartation of resistance to oxidative stress. Therefore, the present invention can be expected to improve conditions that are treatable by, for example, eliminating reactive oxygen species or imparting resistance to oxidative stress by Nrf2 activation. Accordingly, the condition treatable by Nrf2 activation may broadly encompass conditions that are treatable by, for example, eliminating reactive oxygen species or imparting resistance to oxidative stress. More specifically, examples of the condition treatable by Nrf2activation include lifestyle-related diseases, such as diabetes; inflammation; Friedreich's ataxia; cancer; inflammation; arteriosclerosis; hypertension; liver dysfunction; and skin disorders. In addition, embodiments of the present invention may also encompass cosmetic applications. Therefore, the “treatment of a condition treatable by Nrf2 activation” in the present invention also encompasses, for example, skin whitening and prevention or improvement of skin wrinkles.

In the present specification, the “treatment agent for treating a condition treatable by Nrf2 activation of the present invention” may sometimes be simply referred to as the “treatment agent of the present invention.” In the present invention, “treatment of diabetes” encompasses not only lowering the blood glucose levels of patients diagnosed with diabetes, but also supporting the normalization of elevated fasting blood glucose levels, lowering the blood glucose levels of individuals with higher blood glucose levels, and the like. In the present invention, a higher blood glucose level refers to, for example, a fasting blood glucose level of 100 mg/dL or more (for example, a fasting blood glucose level of 110 to 125 mg/dL, or 126 mg/dL or more).

In the present invention, olive refers to plants belonging to the genus Olea of the Oleaceae family, and the type thereof is not particularly limited. Examples of the parts of the olive to be extracted include, but are not limited to, leaves, fruit, stems, and roots, with leaves being preferred. In the present invention, when olive leaves are used, i.e., when a “pressurized hot water extract of olive leaves” is used, the material that is to be extracted and that serves as the raw material of the extract may be any material that mainly contains olive leaves, and may encompass, of course, a material consisting of olive leaves, as well as materials such as olive leaves with portions of branches attached, and branches with multiple olive leaves attached. Therefore, in the present invention, the term “olive leaves” encompasses not only olive leaves alone, but also olive leaves attached to branches and the like, as mentioned above.

The shape and size of olive that is used in this embodiment are not particularly limited as long as the olive can be accommodated within the storage space of the pressure vessel of the extraction apparatus described below.

For example, the olive may be used as is as harvested (i.e., the leaves, fruit, stems, or roots in their natural state), or may be used after being subjected to processing, such as cutting or pulverization, into smaller pieces (for example, into powder). In the latter case, it is possible to improve the processing time and processing efficiency. A known method can be used for reducing the size of olive. For example, olive can be supplied as a pulverized product in the form of chips or powder by performing processing, such as cutting or pulverization.

It is also possible to use olive in a squeezed state using a squeezer or the like. Specifically, the squeezed olive and/or the juice collected after squeezing may be supplied to the pressure vessel of the apparatus mentioned above. In this case, an improvement in the recovery rate of the extract can be expected.

The state of olive is also not particularly limited. Specifically, olive may be used in a state containing water, or in a dried state in which water has been removed to some extent.

In particular, the production method preferably comprises a step of inactivating enzymes present in olive before the olive is placed in the pressure vessel of an extraction apparatus. By providing the step of inactivating enzymes (which is simply referred to below as an “enzyme inactivation step”), enzymatic decomposition of components present in olive leaves can be suppressed. In this case, the influence of enzymes contained in olive can be suppressed; therefore, the production rate of target components (such as hydroxytyrosol and hydroxytyrosol acetate) included in the extract can improve.

The enzyme inactivation step may be performed by any method that is capable of inactivating enzymes present in the olive described above. Examples of such methods include a method of heating olive at a temperature equal to or above the enzyme inactivation point, and a method of drying olive until the enzymes are inactivated. For such drying, not only heat drying but also freeze-drying and the like can be used.

The cultivation conditions of the olive to be extracted is also not particularly limited. For example, it is preferable to use, for example, leaves attached to olive branches pruned during olive fruit harvesting and intended for disposal, in view of the effective use of unused biomass resources.

Olive Extract Obtained by Pressurized Hot Water Extraction In a typical embodiment, the pressurized hot water extract of olive, which is the active ingredient of the present invention, can be obtained by a step of subjecting a mixture containing olive and an extraction solvent, such as water, to a pressurized hot water extraction process.

Specifically, the pressurized hot water extraction process comprises heating the extraction solvent so that the temperature of the mixture containing olive and the extraction solvent, such as water, exceeds 100° C., and extracting water-soluble and fat-soluble components contained in the raw material or components produced from these components, under pressurized conditions.

In a typical embodiment, in the production method, an extraction apparatus equipped with a pressure vessel is used in the step in which the pressurized hot water extraction process is performed.

The pressure vessel of the extraction apparatus has a storage space therein capable of accommodating the extraction solvent and the olive to be extracted, and is configured to enable heating of the mixture contained within the storage space. The temperature within the pressure vessel can be controlled by the extraction apparatus. That is, the extraction apparatus is capable of processing the mixture containing olive and the extraction solvent accommodated within the storage space of the pressure vessel under predetermined high-temperature and high-pressure conditions. The extraction apparatus is not particularly limited as long as it has the functions described above, and may be, for example, a known apparatus.

### Extraction Temperature

In the production method, the extraction temperature for obtaining an extract from the mixture (a mixture containing olive and an extraction solvent) is adjusted to be higher than 100° C. The temperature is preferably 150° C. or higher, more preferably 160° C. or higher, more preferably 170° C. or higher, more preferably 180° C. or higher, more preferably 190° C. or higher, more preferably 200° C. or higher, more preferably 210° C. or higher, and more preferably 220° C. or higher. The upper limit of the extraction temperature is not limited and is preferably 300° C. or lower, and more preferably 250° C. or lower.

By increasing the extraction temperature, the components of olive leaves are more easily dissolved in the extraction solvent in the mixture. More specifically, an increase in the extraction temperature can improve the solubility of the target components, thereby increasing the concentration of the target components in the extract. Furthermore, by increasing the extraction temperature, there is a tendency to reduce the presence of unnecessary components (components other than the target components, which are referred to below as “impurities”). It is presumed that this phenomenon occurs because the impurities are further decomposed by high-temperature processing and become insoluble in water. However, in view of suppressing the hydrolysis of Nrf2-activating components, it is also preferable to adjust the extraction temperature to be lower than a specific temperature. It is known that some components in olive undergo hydrolysis; nevertheless, it has been unexpectedly found that the extract obtained by increasing the extraction temperature within the above ranges has a higher ability to activate Nrf2. From these viewpoints, it is preferable that the extraction temperature is set within the above ranges.

The extraction solvent for use is preferably water. In an embodiment in which water is used as the extraction solvent, an extract containing the target components can be used as is. For example, in this embodiment, an extract with a high degree of safety can be easily prepared. Moreover, this embodiment, in which water is used as the extraction solvent, is advantageous because the potential impact on humans caused by organic solvents etc. can be reduced compared to conventional techniques.

### Extraction Pressure

In the extraction conditions according to a typical embodiment, the extraction pressure can be obtained as a theoretical value corresponding to the extraction temperature. The theoretical value can be determined as a pressure at each extraction temperature based on the vapor-pressure curve of water. For example, when the extraction temperature is 90° C., the extraction pressure is 0.1 MPa, which is the same as atmospheric pressure. When the extraction pressure is 120° C., the extraction pressure is 0.2 MPa. When the extraction temperature is 140° C., the extraction pressure is 0.36 MPa. When the extraction temperature is 200° C., the extraction pressure is 1.55 MPa. Specifically, the inside of the vessel is pressurized due to the increase in the vapor pressure of water caused by heating. Therefore, in a preferred embodiment, extraction is performed without applying additional pressure beyond the vapor pressure of water. Therefore, without particular limitation, the extraction pressure is preferably greater than 0.1 MPa and 5.0 MPa or less, and more preferably greater than 0.5 MPa and 3.5 MPa or less. The theoretical value of the extraction pressure can be calculated from the vapor-pressure curve of the extraction solvent, such as water; however, in this embodiment, since the vapor pressure of volatile components etc. from olive is added, in addition to the vapor pressure of the extraction solvent, the extraction pressure will be greater than the vapor pressure of the extraction solvent.

In the extraction conditions according to this embodiment, the extraction time is not particularly limited as long as the target components can be extracted, and may be suitably adjusted according to the extraction temperature and extraction pressure.

For example, the extraction time is 10 minutes or more, and more preferably 60 minutes or more. Although the upper limit is not particularly limited, performing extraction for 120 minutes or more tends to result in no further improvement in the recovery rate of the extract.

Therefore, from the viewpoint of workability and efficiency, the extraction time is preferably 10 minutes or more and 120 minutes or less, and more preferably 60 minutes or more and 120 minutes or less. The extraction time does not include the period required to reach the predetermined extraction temperature.

For example, when the extraction temperature is 180° C. and the extraction pressure is 1.00 MPa, the extraction time can be adjusted to be 60 minutes or more.

Below, an outline of an extraction apparatus according to one embodiment will be explained. A sealable pressure vessel is equipped with a heating heater, and inside the pressure vessel, a stirrer for improving processing efficiency, a thermometer for monitoring the temperature inside the vessel, and a pressure gauge are provided. A nitrogen gas injection line for injecting nitrogen gas may be connected to the pressure vessel, and a cooling water tank for cooling may be provided outside the pressure vessel.

An example of a step of processing a mixture using the extraction apparatus configured as described above will be explained here. A raw material and an appropriate amount of solvent are placed in the pressure vessel, and the vessel is sealed. Subsequently, the mixture is stirred with the stirrer, and while monitoring the temperature with the thermometer, the mixture is heated to a predetermined temperature and pressure with the heating heater. When the pressure vessel is sealed and the internal air is heated to cause it to expand by the heating heater, the vessel becomes pressurized. It is also possible to inject nitrogen gas into the pressure vessel via the nitrogen gas injection line to increase the initial pressure within the vessel in advance. By increasing the internal pressure, the boiling of the mixture at the inner wall surface of the heated pressure vessel can be prevented. After the temperature reaches a predetermined temperature and/or lapse of time, the entire pressure vessel is cooled. After confirming that the internal temperature of the pressure vessel has sufficiently decreased by cooling, the lid of the pressure vessel is removed and the processed product is collected. The extraction apparatus may also be of a continuous type in which the introduction of the raw material and the collection of the extract are performed continuously.

The pressure vessel after the extraction process according to this embodiment contains therein a liquid extract and residues, such as components insoluble in the extraction solvent and the olive after the extraction process.

The mixture after the extraction process (mixture of the extract and residues) can be separated through processing, such as filtration or centrifugation. The separated extract may be used as is since the extraction solvent is water as described above, or may be used after concentration or the like. In the present invention, the “pressurized hot water extract of olive” encompasses not only the liquid extract mentioned above but also a freeze-dried product of the liquid extract, a pulverized product of the freeze-dried product, and the like.

In this embodiment, the mixing ratio of the olive to the extraction solvent accommodated in the pressure vessel of the extraction apparatus is not particularly limited.

For example, the ratio may be adjusted such that the amount of the extraction solvent is 100 parts by mass or more relative to 100 parts by mass of the olive to be extracted. The amount of the extraction solvent is preferably adjusted to 300 parts by mass or more, and more preferably 500 parts by mass or more.

In the mixing ratio above, increasing the proportion of the extraction solvent can improve extraction efficiency, but reduces the concentration of the target components. Therefore, the upper limit of the amount of the extraction solvent is preferably 2000 parts by mass or less, and more preferably 500 parts by mass or less, relative to 100 parts by mass of the olive. For example, the ratio can be adjusted so that the pressure vessel contains 50 mL of the extraction solvent per 10 g of the olive. In this case, the amount of the extraction solvent is adjusted to 500 parts by mass relative to 100 parts by mass of the olive.

In the production method, the extraction solvent supplied to the pressure vessel of the extraction apparatus is not limited to water alone. An acid, such as citric acid, succinic acid, gluconic acid, or acetic acid, may be added to water, which serves as the extraction solvent. By adding such an acid, the pH of the solvent during extraction can be adjusted. The amount of the acid added is preferably adjusted such that the pH of the solvent before mixing with the olive and before extraction is about 1 to 4, and more preferably about 2 to 3. On the other hand, in embodiments of cosmetics, using water as the extraction solvent is advantageous since it causes less skin irritation compared to solvents such as alcohol.

The treatment agent for a condition treatable by Nrf2 activation according to the present invention encompasses pharmaceuticals, food and beverages, cosmetics, and the like. In the present invention, food and beverages also encompass food with health claims (food with nutrient function claims, food for specified health uses, and food with function claims) and the like.

In the present invention, the treatment agent of the present invention may be a pressurized hot water extract of olive itself or a composition in which the extract is combined with various carriers (such as excipients, tonicity agents, stabilizers, pH adjusters, antioxidants, solubilizing agents, thickeners, and preservatives) that can be added to food and beverages, pharmaceuticals, cosmetics, and the like.

Examples of excipients include maltitol, lactose, lactose monohydrate, mannitol, glucose, microcrystalline cellulose, starch, cyclodextrin, and calcium carbonate. Examples of tonicity agents include sugars, such as glucose, trehalose, lactose, fructose, mannitol, xylitol, and sorbitol; polyhydric alcohols, such as glycerin, polyethylene glycol, and propylene glycol; and inorganic salts, such as sodium chloride, potassium chloride, and calcium chloride.

Examples of chelating agents include edetates, such as disodium edetate, calcium disodium edetate, trisodium edetate, tetrasodium edetate, and calcium edetate; ethylenediaminetetraacetic acid salts, nitrilotriacetic acid or salts thereof, sodium hexametaphosphate, and citric acid.

Examples of stabilizers include sodium bisulfite.

Examples of pH adjusters include acids, such as hydrochloric acid, carbonic acid, acetic acid, and citric acid. Examples also include alkali metal hydroxides, such as sodium hydroxide and potassium hydroxide; alkali metal carbonates or bicarbonates, such as sodium carbonate; alkali metal acetates, such as sodium acetate; alkali metal citrates, such as sodium citrate; and bases, such as trometamol.

Examples of preservatives include sorbic acid; potassium sorbate; parahydroxybenzoate esters, such as methyl parahydroxybenzoate, ethyl parahydroxybenzoate, propyl parahydroxybenzoate, and butyl parahydroxybenzoate; quaternary ammonium salts, such as chlorhexidine gluconate, benzalkonium chloride, benzethonium chloride, and cetylpyridinium chloride; alkylpolyaminoethylglycine; chlorobutanol; polyquad; polyhexamethylene biguanide; and chlorhexidine.

Examples of antioxidants include sodium bisulfite, dried sodium sulfite, sodium metabisulfite, and concentrated mixed tocopherol.

Examples of solubilizing agents include sodium benzoate, glycerin, D-sorbitol, glucose, propylene glycol, hydroxypropyl methylcellulose, polyvinylpyrrolidone, macrogol, and D-mannitol.

Examples of thickeners include polyethylene glycol, methyl cellulose, ethyl cellulose, carmellose sodium, xanthan gum, sodium chondroitin sulfate, hydroxyethyl cellulose, hydroxypropyl cellulose, hydroxypropyl methyl cellulose, polyvinylpyrrolidone, and polyvinyl alcohol.

In an embodiment of the composition, the content of the pressurized hot water extract of olive in the composition is not particularly limited and may be appropriately set from the conditions, such as 99 mass % or more, 95 mass % or more, 90 mass % or more, 70 mass % or more, 50 mass % or more, 30 mass % or more, 10 mass % or more, 5 mass % or more, and 1 mass % or more, in terms of the mass of the solids from the pressurized hot water extract of olive.

The treatment agent of the present invention may further comprise components that are other than the pressurized hot water extract of olive and that can be used for the treatment of a condition treatable by Nrf2 activation. Examples of such components include antidiabetic agents, such as vildagliptin, metformin hydrochloride, alogliptin benzoate, alogliptin, sitagliptin phosphate hydrate, ipragliflozin, L-proline derivatives, and combinations thereof; anticancer agents, such as chemotherapeutic agents, endocrine therapeutic agents, and molecularly targeted agents; and anti-inflammatory agents, such as felbinac, loxoprofen sodium hydrate, methyl salicylate, and tranexamic acid. Examples of components that can be used for the treatment of a condition treatable by Nrf2 activation include whitening components, such as vitamin C derivatives, placenta extract, arbutin, tranexamic acid, and hydroquinone; and wrinkle improvement components, such as retinol, niacinamide, and NEI-L1.

The dosage form is not particularly limited and may be selected from various dosage forms. Examples of the dosage form include orally administered agents, such as solutions, capsules, tablets, pills, powders, granules, and syrups; and non-orally administered agents, such as injections (such as intramuscular injection, intravenous injection, and local injection), gargling solutions, infusion fluids, topical agents (ointments, creams, patches, inhalants), and suppositories.

The treatment agent of the present invention may be administered to subjects, such as mammals. Examples of mammals include humans, monkeys, mice, rats, cats, dogs, pigs, rabbits, cattle, horses, and sheep.

Examples of food and beverages include supplements (e.g., liquids, tablets, and powders); food, such as chocolate and yogurt; and beverages, such as tea drinks and sports drinks.

Examples of cosmetics include skincare cosmetics, such as creams, emulsions, ointments, gels, lotions, and skin toners.

### Nrf2 Activator

As described above, the pressurized hot water extract of olive has an Nrf2 activation ability. Therefore, in one embodiment, the present invention provides an Nrf2 activator comprising a pressurized hot water extract of olive. The details, such as the production method and the method of use for the pressurized hot water extract of olive according to this embodiment, are as described above.

In another embodiment, the pressurized hot water extract of olive, which is the active ingredient of the present invention, can also enhance Nrf2 activation when added to a biological sample, such as cells or organelles, in ex vivo conditions. Therefore, the present invention also provides a method for activating Nrf2, comprising a step of allowing the pressurized hot water extract of olive to act on a biological sample in ex vivo conditions. In this embodiment, examples of the biological sample include those derived from humans, mice, rats, guinea pigs, pigs, monkeys, dogs, cats, African clawed frogs, and the like. The step of allowing the pressurized hot water extract of olive to act on a biological sample may be performed, for example, by adding the pressurized hot water extract of olive to the biological sample. The amount of the pressurized hot water extract of olive to be added is not particularly limited. For example, the amount may be such that the final concentration in the reaction system is 1 to 200 μg/mL, and preferably 10 to 150 μg/mL.

In the method of the present invention, the biological sample, such as cells, mentioned above may be used in a state in which it is placed in a solution, such as a liquid medium. The medium for use here may be appropriately selected from media commonly used for culturing animal cells. Examples of such media include DMEM (Dulbecco's Modified Eagle Medium), RPMI (Roswell Park Memorial Institute) 1640 medium, MEM (Minimum Essential Medium), and DMEM/F12 (Dulbecco's Modified Eagle Medium/Nutrient Mixture F-12) medium. To the media, serum, buffering agents, antibiotics, amino acids, vitamins, and the like may be appropriately added.

Further, in this step, the cells may be maintained in the presence of the pressurized hot water extract of olive. In such an embodiment, while being maintained, the cells may be left to stand or may be shaken. The maintenance time is not particularly limited and may be set, for example, in the range of 1 to 120 hours, and preferably 36 to 72 hours. The temperature in this step is also not limited and may be set, for example, in the range of 30 to 38° C., and preferably 36 to 38° C. By this step, Nrf2 in the biological sample is activated. The activation of Nrf2 can be measured and evaluated according to methods used in the technical field to which the present invention pertains; for example, the measurement and evaluation can be performed by using the methods described in the Examples below.

### Production Method for Treatment Agent and Nrf2 Activator

As described above, the pressurized hot water extract of olive can be obtained by a method comprising a step of subjecting a mixture of olive and an extraction solvent to pressurized hot water extraction. Therefore, in one embodiment, the present invention provides a method for producing a treatment agent for a condition treatable by Nrf2 activation, the method comprising a step of subjecting a mixture of olive and an extraction solvent to pressurized hot water extraction. In another embodiment, the present invention provides a method for producing an Nrf2 activator, the method comprising a step of subjecting a mixture of olive and an extraction solvent to pressurized hot water extraction. The details of the pressurized hot water extraction step and other steps for obtaining the extract (for example, the method for processing olive, i.e., the raw material) are as described above.

Specific embodiments of the present invention will be described in more detail below with reference to Examples; however, the present invention is not limited to these Examples.

### EXAMPLES

### Preparation of Raw Material 1

Dried olive leaves (cultivar: Mission, collected and dried on Feb. 21, 2023, produced by Mitoyo Olive Co., Ltd.) were pulverized using a hammer crusher (NH-34C, Sansho Industry Co., Ltd.), and the olive leaf powder that passed through a 0.7 mm mesh screen was used for extraction processing.

### Test Example 1

The olive leaf powder (100 g) and ion-exchanged water (300 g) were placed in the pressure vessel of a high-pressure microreactor (MMJ-500, produced by OM Labotech Co., Ltd.). Thereafter, the mixture was heated in a sealed state until the liquid temperature reached 220° C., and maintained at 220° C. for 15 minutes (pressure: about 2.6 MPa). The sealed vessel was then cooled to 50° C. to perform pressurized hot water extraction. After the extraction process, the extraction residues (solids) were removed by filtration to obtain a pressurized hot water extract liquid 1.

Subsequently, freeze-drying was performed by using a freeze dryer (FDU-2110, DRC-1000, produced by Tokyo Rikakikai Co., Ltd.) at −30 to-25°C., and the resulting freeze-dried product was pulverized to obtain a pressurized hot water extract 1.

### Test Example 2

The olive leaf powder (5 g) and an ethanol solvent (ethanol:water=8:2) (100 mL) were placed in a beaker, stirred at room temperature overnight, and subjected to extraction. After the extraction process, the extraction residues (solids) were removed by filtration to obtain an ethanol extract liquid. Subsequently, the ethanol extract liquid was concentrated and dried by using an evaporator (N-1000, NVP-2100, NVC-3000, produced by Tokyo Rikakikai Co., Ltd.), and then pulverized to obtain an ethanol extract.

### Test Example 3

The olive leaf powder (5 g) and water (100 mL) were placed in a beaker, stirred at 90° C. for two hours, and subjected to hot water extraction. After the extraction process, the extraction residues (solids) were removed by filtration to obtain a hot water extract liquid.

Subsequently, the hot water extract liquid was freeze-dried at −30 to −25°C. by using a freeze dryer (FDU-2110, DRC-1000, produced by Tokyo Rikakikai Co., Ltd.), and the resulting freeze-dried product was pulverized to obtain a hot water extract.

### Preparation of Raw Material 2

Dried olive leaves (cultivar: Mission, collected and dried on Mar. 13, 2024, produced by Mitoyo Olive Co., Ltd.) were pulverized using a hammer crusher (NH-34C, Sansho Industry Co., Ltd.), and the olive leaf powder that passed through a 0.7 mm mesh screen was used for extraction processing.

### Test Example 4

The olive leaf powder (40 g) and ion-exchanged water (120 g) were placed in the pressure vessel of a high-pressure microreactor (MMJ-500, produced by OM Labotech Co., Ltd.). Thereafter, the mixture was heated in a sealed state until the liquid temperature reached 160° C., and maintained at 160° C. for 60 minutes (pressure: about 0.8 MPa). The sealed vessel was then cooled to 50° C. to perform pressurized hot water extraction. After the extraction process, the extraction residues (solids) were removed by filtration to obtain a pressurized hot water extract liquid 2. Subsequently, the pressurized hot water extract liquid 2 was freeze-dried by using the freeze dryer at −30 to −25°C. as in Test Example 1, and the resulting freeze-dried product was pulverized to obtain a pressurized hot water extract 2.

### Test Example 5

The olive leaf powder (40 g) and ion-exchanged water (120 g) were placed in the pressure vessel of a high-pressure microreactor (MMJ-500, produced by OM Labotech Co., Ltd.). Thereafter, the mixture was heated in a sealed state until the liquid temperature reached 190° C., and maintained at 190° C. for 30 minutes (pressure: about 1.5 MPa). The sealed vessel was then cooled to 50° C. to perform pressurized hot water extraction. After the extraction process, the extraction residues (solids) were removed by filtration to obtain a pressurized hot water extract liquid 3. Subsequently, the pressurized hot water extract liquid 3 was freeze-dried by using the freeze dryer as in Test Example 1, and the resulting freeze-dried product was pulverized to obtain a pressurized hot water extract 3.

### Test Example 6

The olive leaf powder (40 g) and ion-exchanged water (120 g) were placed in the pressure vessel of a high-pressure microreactor (MMJ-500, produced by OM Labotech Co., Ltd.). Thereafter, the mixture was heated in a sealed state until the liquid temperature reached 220° C., and maintained at 220° C. for 15 minutes (pressure: about 2.6 MPa). The sealed vessel was then cooled to 50° C. to perform pressurized hot water extraction. After the extraction process, the extraction residues (solids) were removed by filtration to obtain a pressurized hot water extract liquid 4.

Subsequently, the pressurized hot water extract liquid 4 was freeze-dried using the freeze dryer as in Test Example 1, and the resulting freeze-dried product was pulverized to obtain a pressurized hot water extract 4.

### Example 1

The evaluation of Nrf2 activation was performed by using HepG2 human hepatocellular carcinoma-derived cell lines into which a construct containing red-emitting luciferase SLR3 fused downstream of the antioxidant response element (ARE), i.e., the Nrf2 response element, and a construct containing a green-emitting luciferase ELuc fused to the thymidine kinase promoter (a total of two types of constructs) were stably incorporated. The HepG2 cells were seeded into a 96-well plate at 3×10⁴ cells per well and cultured overnight in a CO₂ incubator. The medium was then replaced with a medium containing a predetermined concentration of the pressurized hot water extract of olive leaves (Test Example 1), the 80% ethanol extract, or the hot water extract, and luminescence was measured in real time at about 30-minute intervals over a period of 72 hours using a microplate-type real-time luminometer (Kronos-HT, ATTO Corporation).

Subsequently, the fold change value at each measurement point was calculated according to the following formula:

Fold change value=(Luminescence intensity of SLR3 in sample-treated group/Luminescence intensity of ELuc in sample-treated group)/(Luminescence intensity of SLR3 in vehicle-treated group/Luminescence intensity of ELuc in vehicle-treated group).

The Nrf2 activation observed in each extract was evaluated based on the area under the curve (AUC) of fold change values over a course from 0 to 72 hours (upper row). The effect on cell viability was evaluated based on the AUC of the green-emitting luciferase ELuc measured from 36 to 72 hours (lower row).

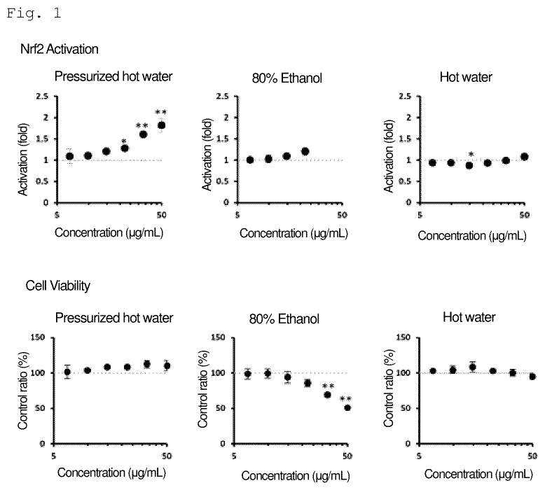

FIG. 1 shows the results. As shown in FIG. 1, the results clarified that only the pressurized hot water extract (Test Example 1) activated Nrf2 in a concentration-dependent manner without showing cytotoxicity. In terms of the 80% ethanol extract, since the cell viability was 70% or less at test concentrations of 50 and 33 μg/mL, the data for Nrf2 activation at these concentrations were excluded, and the values measured at 7 to 22 μg/mL were used. The results clarified that the 80% ethanol extract does not activate Nrf2 at concentrations that are non-cytotoxic. The hot water extract showed neither Nrf2 activation nor cytotoxicity at any of the tested concentrations.

### Example 2

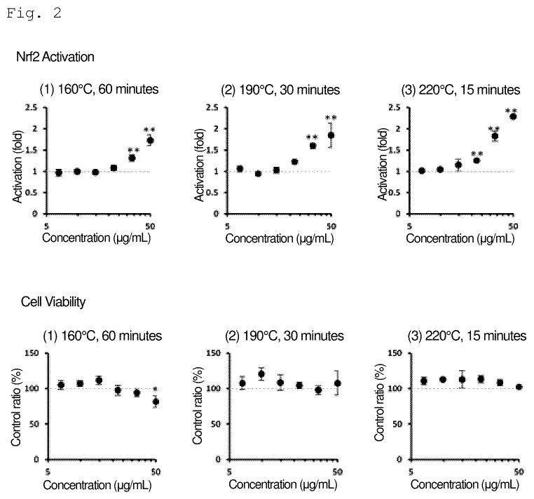

In accordance with the same measurement method as in Example 1, the Nrf2 activation (upper row) and the cell viability (lower row) of the pressurized hot water extracts of olive leaves obtained under different extraction conditions ((1) 160° C. for 60 minutes (Test Example 4), (2) 190° C. for 30 minutes (Test Example 5), and (3) 220° C. for 15 minutes (Test Example 6)) were evaluated. FIG. 2 shows the results. As shown in FIG. 2, the results clarified that in all of the extraction conditions, Nrf2 activation was observed in a concentration-dependent manner without showing cytotoxicity. The results also clarified that Nrf2 activation increased as the extraction temperature increased.

### Example 3

The main components in the pressurized hot water extract of olive leaves (Test Example 1), the 80% ethanol extract, and the hot water extract were quantified by high-performance liquid chromatography (HPLC) under the following conditions:
Column: Capcell Pak UG 120 C18 column (5 μm, 4.6×250 mm, Osaka Soda Co., Ltd.); Flow rate: 1.0 mL/min; Mobile phase: (A) water containing 0.1% trifluoroacetic acid, (B) acetonitrile containing 0.1% trifluoroacetic acid; Gradient: A:B=95:5 (0-20 min)−85:15 (20.1 min)−50:50 (45 min)−0:100 (45.1-55 min); Detection wavelength: 280 nm. The quantified compounds were each quantitatively analyzed using commercially available standard products, and calibration curves were created. The quantification limit and detection limit for each compound were defined based on signal-to-noise ratios of 10 and 3, respectively.

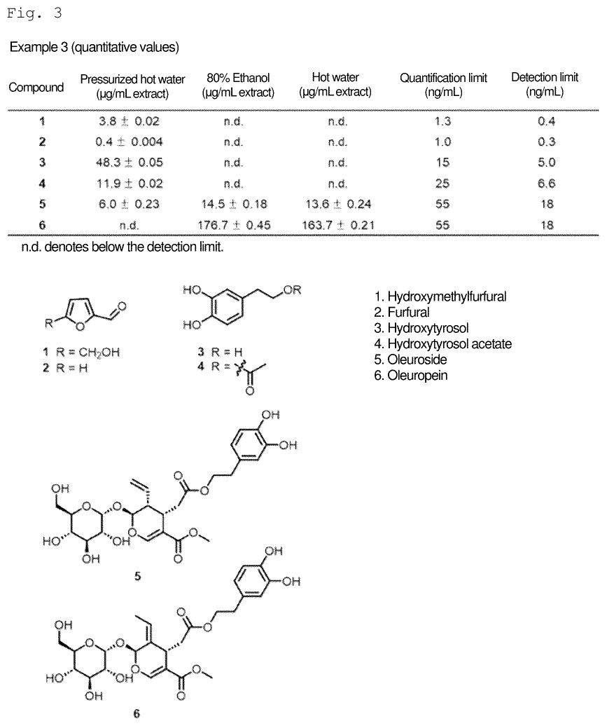

FIG. 3 shows the results. As shown in FIG. 3, the results clarified that the pressurized hot water extract contained hydroxymethylfurfural (1), furfural (2), hydroxytyrosol (3), hydroxytyrosol acetate (4), and oleuroside (5) as main components. The results also revealed that the 80% ethanol extract and the hot water extract contained oleuroside (5) and oleuropein (6) as main components.

### Example 4

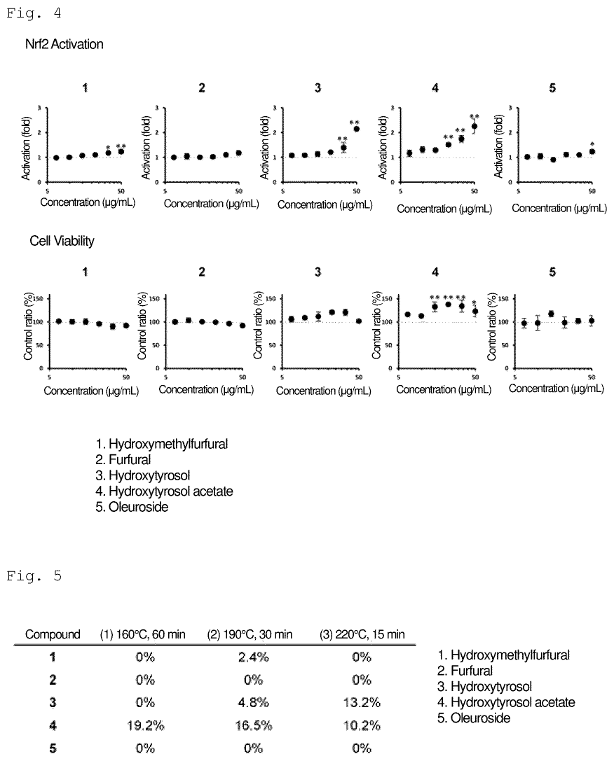

In accordance with the same measurement method as in Example 1, the Nrf2 activation (upper row) and the cell viability (lower row) of compounds 1 to 5 contained in the pressurized hot water extract of olive leaves were evaluated. FIG. 4 shows the results. As shown in FIG. 4, the results clarified that hydroxytyrosol (3) and hydroxytyrosol acetate (4) activated Nrf2 in a concentration-dependent manner without showing cytotoxicity. Hydroxymethylfurfural (1), furfural (2), and oleuroside (5) showed neither Nrf2 activation nor cytotoxicity at any concentration.

### Example 5

The contribution rates, to Nrf2 activation, of compounds contained in three types of the pressurized hot water extracts of olive leaves obtained under different extraction conditions ((1) 160° C. for 60 minutes (Test Example 4), (2) 190° C. for 30 minutes (Test Example 5), and (3) 220° C. for 15 minutes (Test Example 6)) were compared.

The contribution rates were calculated as follows: (i) the concentration of each compound contained in the extracts of Test Examples 4 to 6 was measured (data not shown); (ii) from FIG. 4, a regression equation was created for each compound to represent the relationship between Nrf2 activation and compound concentration, and (iii) the data measured in (i) were entered into the regression equations, and the Nrf2 activation value of each compound in the extracts of Test Examples 4 to 6 at 50 μg/mL was calculated. Subsequently, from FIG. 2, the Nrf2 activation value of the pressurized hot water extract of olive leaves at 50 μg/mL was calculated, and the contribution rates were calculated according to the following formula:

Contribution rate (%)=(Nrf2 activation value of each compound−1)/(Nrf2 activation value of pressurized hot water extract of olive leaves−1)×100).

FIG. 5 shows the results. As shown in FIG. 5, the contribution rates of the compounds contained in each extract to Nrf2 activation were about 20% when hydroxytyrosol (3) and hydroxytyrosol acetate (4) were considered together. Therefore, only about 20% of the Nrf2 activation observed in the pressurized hot water extract of olive leaves was confirmed to be attributable to hydroxytyrosol and hydroxytyrosol acetate contained in the pressurized hot water extract of olive leaves. That is, the Nrf2 activation ability of the pressurized hot water extract of olive leaves greatly exceeded the level expected from the Nrf2 activation of hydroxytyrosol and hydroxytyrosol acetate.

Furthermore, as stated above, in Example 2, the pressurized hot water extracts obtained at higher extraction temperature tended to show higher Nrf2 activation ability. On the other hand, as shown in FIG. 5, the contribution of hydroxytyrosol and hydroxytyrosol acetate to Nrf2 activation remained unchanged at about 20% in total, regardless of the heating temperature.

These results suggested the possibility that other components that were not detected in the analysis of FIG. 3 may also contribute to Nrf2 activation observed in the extract.

1. A method for treating a condition treatable by Nrf2 activation, comprising administering an effective amount of a pressurized hot water extract of olive to a subject in need thereof.

2. A method for activating Nrf2, comprising administering an effective amount of a pressurized hot water extract of olive to a subject in need thereof.

3. The method according to claim 1, wherein the pressurized hot water extract of olive comprises a pressurized hot water extract of olive leaves.

4. The method according to claim 1, wherein the extract is obtained by subjecting olive to pressurized hot water extraction at a temperature of 150°C. or higher.

5. The method according to claim 4, wherein in the pressurized hot water extraction, the extraction pressure is greater than 0.1 MPa and 5.0 MPa or less, and the extraction time is 120 minutes or less.

6. A method for producing a treatment agent for a condition treatable by Nrf2 activation, the method comprising a step of subjecting a mixture of olive and an extraction solvent to pressurized hot water extraction.

7. A method for producing an Nrf2 activator, comprising a step of subjecting a mixture of olive and an extraction solvent to pressurized hot water extraction.

8. The production method according to claim wherein the olive is an olive leaf.

9. The production method according to claim 6, wherein the pressurized hot water extraction step is performed at a temperature of 150°C. or higher.

10. The production method according to claim 9, wherein the pressurized hot water extraction step is performed at a pressure greater than 0.1 MPa and 5.0 MPa or less for 120 minutes or less.
